(19) United States
(12) Patent Application Publication
(10) Pub. No.: US 2020/0268651 A1
(43) Pub. Date: Aug. 27, 2020

(54) STABLE VITAMIN B12 LIQUID FORMULATIONS

(71) Applicants: Hemant N. Joshi, Parsippany, NJ (US); Neelam Sharma, Morris Plains, NJ (US)

(72) Inventors: Hemant N. Joshi, Parsippany, NJ (US); Neelam Sharma, Morris Plains, NJ (US)

(21) Appl. No.: 16/282,713

(22) Filed: Feb. 22, 2019

(51) Int. Cl. A61K 9/00; A61K 9/48; A61K 9/50; A61K 31/155; A61K 31/55; A61K 31/341; A61K 31/714

(52) U.S. Cl. CPC A61K 9/0056; A61K 9/4816; A61K 9/4833; A61K 9/4825; A61K 9/5036; A61K 9/485; A61K 9/5015; A61K 9/0043; A61K 31/155; A61K 31/55; A61K 31/341; A61K 31/714; A61K 9/0019

(57) ABSTRACT

An aqueous solution of vitamin B12 can be stabilized by the synergistic effect of HPMC, carrageenan and potassium acetate. This property is used to develop four formulations—1. A solution used to prepare B12 loaded capsule shell matrix, 2. vitamin B12 injectable formulation, 3. vitamin B12 nasal formulation and 4. Vitamin B12 oral solution formulation.

### RELATED APPLICATIONS

This patent describes stabilization of vitamin B12 liquid formulations. The approach is used to develop vitamin B12-loaded capsule shell matrix, injectable formulation, nasal spray and oral liquid. The concept of vitamin B12-loaded capsule shell matrix is related US Patent application #2018/0116970 filed on Dec. 30, 2017 and US patent application #20180318228 filed on Jun. 11, 2018. The current patent application is not claiming benefit of these patents or patent applications.

### FIELD OF INVENTION

The present invention relates to stable liquid formulations of vitamin B12. We are claiming the compositions of improved formulations of vitamin B12.

### BACKGROUND OF THE INVENTION

Vitamin B₁₂, also called cobalamin, is a water-soluble vitamin that is involved in the metabolism of every cell of the human body: it is a cofactor in DNA synthesis (it is required for certain enzymes to function), and in both fatty acid and amino acid metabolism. It is particularly important in the normal functioning of the nervous system via its role in the synthesis of myelin, and in the maturation of developing red blood cells in the bone marrow. Vitamin B12 is essential for nerve conduction, mental functioning, DNA synthesis, and red blood cell formation. Vitamin B12 contains rare element cobalt positioned in the center of the corrin ring. It is available in animal products such as meat, fish, eggs, milk and milk products, but it is not present in many plants. Vitamin B12 is also available in Soy products, almonds, oat and rice beverage (Canadian nutrient file 2015). Thus, people having vegan diet should take vitamin B12 from an external source.

There are four vitamers of B12—cyanocobalamin, hydroxocobalamin, adenosylcobalamin and methylcobalamin. For the purpose of this patent, term vitamin B12 refers to any of these four vitamers of B12.

Vitamin B12 functions as a coenzyme. It means, its presence is mandatory for enzyme-catalyzed reactions. There are three types of enzymes, which need vitamin B12—isomerases, methyltransferases and dehalogenases.

Vitamin B12 can be converted to coenzyme B12 in tissues, and as such is essential for conversion of methylmalonate to succinate and synthesis of methionine from homocysteine, a reaction which also requires folate. In the absence of coenzyme B12, tetrahydrofolate cannot be regenerated from its inactive storage form, 5-methyltetrahydrofolate, and a functional folate deficiency occurs. Vitamin B12 also may be involved in maintaining sulfhydryl (SH) groups in the reduced form required by many SH-activated enzyme systems. Through these reactions, vitamin B12 is associated with fat and carbohydrate metabolism and protein synthesis (Nascobal® cyanocobalamin nasal spray package insert).

Causes for the Deficiency of Vitamin B12 and Effects:

The gastro-intestinal (GI) absorption of vitamin B12 reduces as we grow old. Intestinal causes such as ilial resection, crohn's disease affecting ileum, and any radiotherapy causing irradiation of ileum can produce vitamin B12 deficiency. Some of the common causes are—1. Autoimmune disease which destroy specialized cells in the GI tract. These specialized cells help/promote vitamin B12 absorption, 2. Small intestinal bacterial overgrowth, 3. Inflammation in the tail-end of the small intestine, and 4. Surgery that removes part of small intestine, particularly ileum. Some drugs such as colchicine, neomycin, metformin and anti-convulsants can also cause vitamin B12 deficiency. Following drugs are also known to impair absorption of vitamin B12—alcohol, antibiotics, gastric acid inhibitor, a biguanide, a proton pump inhibitor, H2 receptor antagonist and many others. Gastric acid is needed to release vitamin B12 from protein for absorption. Thus, H2-receptor antagonists and proton-pump inhibitors, which reduce secretion of gastric acid can reduce the absorption of B12. This condition may be characterized by limb neuropathy or a blood disorder called pernicious anemia.

Long-term use of metformin, a commonly used antihyperglycemic agent, can reduce serum levels of vitamin B12 in up to 30% of people. It is claimed to reduce the absorption of Vitamin B12. Diabetes Prevention Program Outcomes Study (DDPOS) represents one of the largest and longest studies of metformin treatment available. Results from the DDPOS showed that long-term metformin use increased the risk for vitamin B12 levels. It is believed that metformin can affect the absorption of vitamin B12 in the ileum of the small intestine. In another study, it was observed that higher the metformin dose, the more deficient people were in vitamin B12 and that. metformin reduced vitamin B12 levels in both long (≥3 years) and short (<3 years) term use. (Aroda et al., 2016).

Administration of anti-epileptic drugs have been reported to cause vitamin B12 deficiency (Asian K. et al., 2008; Linnebank M. et al., 2011; Huang et al., 2016)

Absorption of Vitamin B12 from the GI Tract:

Vitamin B12 has good water solubility. It has a high molecular weight (cobalamin 1355.4 g/mol). If vitamin B12 is ingested in its free (or nonprotein bound form), it will bind to a carrier protein known as R-binders or transcobalamin I that is secreted by both the salivary glands in the oropharynx and the gastric mucosal cells within the stomach. If the vitamin B12 is ingested in its protein bound form, it must first undergo a proteolytic cleavage in the stomach or duodenum where it binds to a R-binder and enters the duodenum for further cleavage. Upon entry into the second segment of the duodenum, the pancreas secretes additional protease, which then degrades the R-binders holding onto the vitamin B12. It is at this point that vitamin B12 binds to or complexes with intrinsic factor for the remainder of its journey to the ileum of the small intestine for absorption. It is absorbed through a passive diffusion process and actively via intrinsic factor (Jain et al, 2015). Kolber and Houle stated that oral administration of vitamin B12 is accepted by patients and it is cost effective. (Kolber et al., 2014). If patients have malabsorption of vitamin B12, oral administration may show low bioavailability (Castelli et al., 2011)

Absorption of Vitamin 312 from the Nasal Route:

A three-way crossover study in 25 fasting healthy subjects was conducted to compare the bioavailability of the B12 nasal spray to the B12 nasal gel and to evaluate the relative bioavailability of the nasal formulations as compared to the intramuscular injection. The peak concentrations after administration of intranasal spray were reached in 1.25±1.9 hours. The bioavailability of the vitamin B12 nasal spray was found to be 10% less than the Vitamin B12 nasal gel. The bioavailability of the nasal spray relative to the intramuscular injection was found to be 6.1% (Nascobal® package insert).

Sublingual Absorption of Vitamin B12:

Oral cavity is an attractive site due to ease of administration. The buccal administration of drugs can have mucosal (local) effect or transmucosal (systemic) effect. Two review articles describe the concept, advantages and disadvantages of buccal delivery systems (Smart, J. D., 1.993; Chinna R. P. et al., 2011). Strong P and their colleagues showed that decreased serum vitamin B12 level in patients with type 2 diabetes on long-term metformin treatment can be corrected through treatment with vitamin B12 injections or sublingual supplements (Strong et al., 2016). It seems that vitamin B12 is absorbed in the mouth cavity by passive diffusion mechanism. Thus, we need to have a sufficient concentration in the mouth-cavity and it should reside long enough to allow passive absorption of vitamin B12. Thus, a slow-release orally dissolving capsule is supposed to have a better efficacy for vitamin B12 over fast releasing orally disintegrating tablet.

Currently Available B12 Formulation:

There are various types of dosage form available for vitamin B12 as prescription products. Multivitamin solutions, cyanocobalamin (1 mg/mL) injections, and a nasal spray. Also, there was a nasal gel formulation, which has been now discontinued. Vitamin B12 is also available as lyophilized powder for injection. There are several over-the-counter products of vitamin B12 available. These are—methyl cobalamin lozenges (5 mg/lozenge), time-release tablets of methyl cobalamin (1 mg/tablet), methyl cobalamin and hydroxocobalamin gummies (3 mg/unit), quick-dissolve vitamin B12 tablets (2.5 mg/tablet), and softgels (1 mg/softgel).

Patents for B12 Formulations:

Vitamin B12 in a liquid formulation is not stable. Chelating agents, alkali metal salts, cyclodextrins, polyvalent alcohols, dextran, gelatin have been reported to stabilize vitamin B12 (Heep I., US patent 2011/0065665). The inventors also used a mixture of butaphosphan/n-butanol as stabilizing agents for vitamin B12. The same group filed another patent, in which the pH was maintained between 4 to 7 (Heep I. and Taterra H. R., U.S. Pat. No. 9,089,582). McCarty (US patent 2016/0000716) proposed a method of treating B12 deficiency by a sublingual or buccal administration of B12 tablet formulation. The formulation contained propylene glycol to dissolve cobalmin, solid adsorbent, water soluble excipient, a disintegrant and a lubricant. US patent 2008/0039422 (Cruz A. and Pristupa Z.) proposed pharmaceutical compositions to treat B12 deficiency. Inventors used excipients to enhance the solubility of vitamin B12 compounds. They used alcohols such as ethanol, propylene glycol, PEG, glycerol, mannitol, sorbitol etc. Goldberg and Arbit (WO/2013/028333) described edible strips to be placed in the oral cavity. They intended to deliver different kinds of active agents including caffeine, vitamin E, Vitamin B12 etc. The patent teaches to make a strip or a film and not a capsule. Gutierrez and Beer (2018/0085310) proposed a dietary supplement in the form of a liquid to improve brain health, which contained vitamin B12.

Capsules are one of the most commonly used pharmaceutical dosage forms, which are easy to manufacture. Capsules are made from aqueous solution of a gelling agent such as animal protein or plant polysaccharide or their derivatives. Capsules are generally of two types: hard and soft capsules. Hard capsules as name suggests are hard for feel and soft being soft. Many specific constituents have been used to form the shell of capsule. One basic component is film forming material, which can be gelatin, hydroxylpropyl methylcellulose (HPMC), hydroxypropyl cellulose, methyl cellulose and mixtures thereof. For the purpose of this patent, these film-forming materials are defined as “Hardshell-forming materials”.

Capsules are readily soluble in water at 37° C. Many types of drug-loaded materials are filled into the capsule shells. Types of materials for filling into hard gelatin capsules are dry solids such as powders, pellets, granules or semisolids such as suspensions, pastes or liquids such as non-aqueous liquids.

There are various sizes of hard capsule shells available ranging from sizes ‘000’ to ‘5’ (higher the number, smaller is the dose volume), the most commonly used are sizes ‘0’ and ‘1’.

A hard-capsule size chart is shown in Table 1. These values may vary for each capsule manufacturer.

TABLE 1
Details on various sizes of capsules including the amount of powder that can be filled.

| Size | 000 | 00el | 00 | 0el | 0 | 1 | 2 | 3 | 4 | 5 |
|---|---|---|---|---|---|---|---|---|---|---|
| Volume, mL | 1.37 | 1.02 | 0.95 | 0.78 | 0.68 | 0.50 | 0.37 | 0.30 | 0.21 | 0.10 |
| Weight empty | 158.1 | 128 | 115.7 | 99.7 | 89.8 | 71.8 | 58.1 | 48.6 | 37.5 | 24.1 |
| capsule (mg) | | | | | | | | | | |
| Density (g/cc) | Powder weight in mg | | | | | | | | | |
| 0.3 | 411 | 306 | 285 | 234 | 204 | 150 | 111 | 90 | 63 | 30 |
| 0.4 | 548 | 408 | 380 | 312 | 272 | 200 | 148 | 120 | 84 | 40 |
| 0.5 | 685 | 510 | 475 | 390 | 340 | 250 | 185 | 150 | 105 | 50 |
| 0.6 | 822 | 612 | 570 | 468 | 408 | 300 | 222 | 180 | 126 | 60 |
| 0.7 | 959 | 714 | 665 | 546 | 476 | 350 | 259 | 210 | 147 | 70 |
| 0.8 | 1096 | 816 | 760 | 624 | 544 | 400 | 296 | 240 | 168 | 80 |
| 0.9 | 1233 | 918 | 855 | 702 | 612 | 450 | 333 | 270 | 189 | 90 |
| 1.0 | 1370 | 1020 | 950 | 780 | 680 | 500 | 370 | 300 | 210 | 100 |
| 1.1 | 1507 | 1122 | 1045 | 858 | 748 | 550 | 407 | 330 | 231 | 110 |
| 1.2 | 1644 | 1224 | 1140 | 936 | 816 | 600 | 444 | 360 | 252 | 120 |
| 1.3 | 1781 | 1326 | 1235 | 1014 | 884 | 650 | 481 | 390 | 273 | 130 |
| 1.4 | 1918 | 1428 | 1330 | 1092 | 952 | 700 | 518 | 420 | 294 | 140 |
| 1.5 | 2055 | 1530 | 1425 | 1170 | 1020 | 750 | 555 | 450 | 315 | 150 |

Currently orally disintegrating tablet or orally dissolving tablet (ODT) are available in the market. They were designed for people suffering for dysphagia who can take the drugs as ODT without water. Now ODT has been used as a new kind of drug delivery system and has been widely accepted. Currently, many drugs are administered as ODT and Table 2 lists few such drugs. Some of the examples of approved orally fast dissolving tablets are—Loratidine, Cetirizine, Cisapride monohydrate, Risperidone, Zolmitriptan, Hyoscyamine Sulfate, Famotidine, Tramadol HCL, Phloroglucinol Hydrate etc. It is evident from Table 2 that ODT's are useful to various age groups. This patent application uses the Orally dissolving capsule (ODC) technology in one of the formulations. The same drugs and many additional drugs can also be delivered using ODC technology.

TABLE 2
List of active pharmaceuticals administered as ODT.

| Intended | |
|---|---|
| Age | Active ingredient |
| 0-5 years | Clonazepam, Iamotrigine, Hyoscyamine Iansoprazole, |
| 5-10 years | Aripriprazole, Amphetamine, Fexofanadine, |
| | Desmopressin, Tramadol |
| 10-15 years | Diphenhydramine, Domperidone, Risperidone, Ibuprofen |
| 15-20 years | Meloxicam, Tramadol, |
| Adults | Selegiline, Olanzapine, Metoclopramide, Famotidine |

U.S. Pat. No. 8,105,625 (Rajewski and Haslam) patent described the formulation of fast dissolving capsules to enhance the therapeutic effect. In this patent, Rajewski and Haslam prepared hard-shell capsules with pullulan, a plasticizer and a dissolution enhancing agent. The capsules were meant to dissolve in the mouth cavity (ODC or orally dissolving capsules).

One of the present patent concepts originally related to the hard-capsule shell composition containing vitamin B12. The capsules are normally prepared using gelatin and other excipients. In recent times, several polymers have been employed to manufacture hard-shell capsules. So far, the capsule core is filled with a drug or combination or drugs, in the form of powder, granules, beads, pellets etc. The “capsule core” is the space within the capsule shells and not the capsule shell composition. Hollow is defined as an empty space. A “hollow core hard-shell capsule” means a hard-shell capsule with an empty space in the core.

One has to be very careful about understanding the definitions of “capsule shell-forming composition” and “core composition”. Gelatin, polyol and water-insoluble component formed a “capsule shell-forming composition” with which, the capsules were formed. The “core composition” is the one which is filled in the above-mentioned capsule shell. The “core composition” may include sweeteners, flavors, medicaments and other excipients. One of the concepts in the current patent application is related two approved patents by the same author (Hemant Joshi), U.S. Pat. Nos. 8,728,521 and 9,884,024. In these, the medicament is either physically/molecularly distributed, and/or chemically bound to the polymer matrix of the capsule shell composition. The concept of current application focused on molecular distribution of vitamin B12 in the capsule shell composition of the hard-shell capsules. There are mainly two methods to prepare capsules: pin dip-coating and heat-melting. This patent uses the pin-dip method. In this, a liquid mass is produced by dissolving the capsule shell-forming compositions in a solvent system. In the pin-dip method, plurality of pins maintained at a certain temperature is dipped in the solution and is withdrawn at a pre-determined rate while spinning. The pins coated with capsule composition are then dried at a gradual rate. The capsules (body and cap) are separated from the pins and are trimmed to an exact length. The method has been employed to prepare the body and cap of the capsules. The body and cap are joined or fitted together or cooperatively engaged and a logo is printed, if necessary.

During the development of this concept, authors discovered stabilization of vitamin B12 solutions, which provided the novelty to this patent application. The stable liquid of vitamin B12 can be used to develop various dosage forms. One such form is a liquid used to prepare capsule shells. The other three could be—injectable formulation, nasal spray and oral liquid.

### SUMMARY OF THE INVENTION

The present invention proposes to a design to incorporate vitamin B12 in the hard-shell capsule matrix (body and/or cap). The concept produces a capsule dosage form in which the active moiety, vitamin B12, is incorporated in the shell matrix to produce an empty hard-shell capsule. There are four vitamers or forms of vitamin B12—cyanocobalamin, hydroxocobalamin, adenosylcobalamin and methyl cobalamin. Thus, vitamin B12 presented refers to any of these vitamers or the mixture thereof. Empty hard-shell capsule forming material in this patent application comprises of gelatin or a suitable polymer or a mixture of polymers, and suitable excipients including plasticizer, coloring agent, sweetener, a preservative including antioxidant etc. The polymers used to make the capsule shell are hydroxypropyl methylcellulose, hydroxypropyl cellulose, methyl cellulose and mixtures thereof.

The present invention proposes stable solution formulations containing vitamin B12. In one embodiment, an aqueous solution of vitamin B12 contained carrageenan. In another embodiment, an aqueous solution of vitamin B12 contained carrageenan and potassium acetate. In yet another embodiment, an aqueous solution of vitamin B12 contained carrageenan, potassium acetate and a cellulosic polymer such as HPMC. For the purpose of this patent, carrageenan term refers to kappa carrageenan, lambda carrageenan and iota carrageenan. These aqueous vitamin B12 solutions also can contain excipients such as a sweetener, a preservative, a flavor etc. One can develop different types of formulations using this basic vitamin B12 liquid composition—1. A liquid or gel to be used to prepared empty capsules with vitamin B12 molecularly embedded in the capsule shell matrix. 2. An injectable vitamin B12 solution formulation 3. Nasal spray or Nasal gel formulation, and 4. An oral B12 liquid formulation. Each of these formulations contain therapeutically effective amount of vitamin B12.

The result of the present invention is a method for the slow release of vitamin B12 from orally dissolving capsules (ODCs). These capsules are prepared using above mentioned stabilized vitamin B12 solution. The capsules couldn't have been prepared without stabilization of Vitamin B12 during the manufacturing process. These ODCs can be administered without coadministration of water or any other liquid. For elderly patients, it is customary to take medication in the evening or before going to bed. If the medications are taken with water, the water intake so late at night may make them use the restroom at night and thereby, disturbing their sleep. In such instances, taking medication without water can be very useful.

In this particular case, another advantage of the invention is to slowly release of vitamin B12 over time in the oral cavity to allow buccal and sublingual absorption (or oral transmucosal absorption) and thereby, reduce hepatic first pass effect at least for a portion of the drug, which is absorbed through the buccal cavity. The main objective of these B12-loaded ODC capsules is to provide a method of administration of ODC to release Vitamin B12 over a long time allowing a sustained release thereby increasing buccal absorption; i.e., increase the residence time of the drug delivery system in the mouth cavity.

The present invention is directed to achieve a novel drug delivery system for ODC, comprising a hard-capsule shell which may contain a sweetener, a sequestering agent, suitable salts, a gelling agent and a flavoring agent or combination thereof. The sweeteners and flavoring agents provide an acceptable taste or mask the taste of the drug, if necessary. The sequestering agent or other salts may induce salivation. Many people suffer from dry mouth and saliva will help them.

In another embodiment of the invention, the empty hard-shell capsule containing vitamin B12 in the capsule shell matrix is administered without any core composition filled in the core of the capsule. Essentially, the capsule core will be empty.

In another embodiment of the invention, the core of the hard-shell capsule containing vitamin B12 in the capsule shell matrix is filled with inert substances such as sugar, lactose, chocolate etc. and mixtures thereof along with flavoring agents.

In another embodiment of the invention, the core of the hard-shell capsule containing vitamin B12 in the capsule shell matrix is filled with a drug or drugs; mainly those drugs, which reduce the absorption of vitamin B12 or cause vitamin B12 deficiency. The capsule filling material containing drug/drugs is in the form of powder, microspheres, granules, beads, pellets, mini-tablets. The core material can be coated with polymer or coating material for slow release, sustained release, time-release, controlled release or modified release action or as an enteric coat. One such drug may be metformin. In another embodiment, these capsules can be swallowed with a liquid, if the patient desires.

Vitamin B12 is known to be light and heat sensitive. The medicament has to be stable in the capsule shell matrix during manufacture and during storage of the product. The product is stored in the refrigerator or at room temperature protect from light. The primary packaging material can be a blister-pack or a colored glass or polymeric bottles. If the primary bottle is not colored, it must be protected from light by a suitable secondary packing. The capsule shell may absorb moisture during storage. Higher moisture level in the hard capsule can make them soft. In the worst-case scenario, capsules can become sticky and adhere to each other in the bottle. Thus, the vitamin B12 loaded empty hard-shell capsules must be protected from moisture and light.

In another embodiment, an injectable formulation is prepared with carrageenan, potassium acetate and a polymer, the polymer can be HPMC. The formulation can be a single-dose or a multi-dose. If it is a single dose formulation, there is no need to add a preservative. If it is a multi-dose formulation, it must contain a preservative such as benzyl alcohol. One such typical composition contains therapeutically effective amount of vitamin B12, sodium chloride, benzyl alcohol, carrageenan, potassium acetate, HPMC, hydrochloric acid and/or sodium hydroxide to adjust the pH between 4.5 and 7.5 and water for injection. The formulation is sterilized by autoclaving. Another composition contains therapeutically effective amount of vitamin B12, sodium chloride, benzyl alcohol, carrageenan, potassium acetate, hydrochloric acid and/or sodium hydroxide to adjust the pH between 4.5 and 7.5 and water for injection. These injectable formulations of vitamin B12 are filled in vials, prefilled syringes, or ampules protected from light. The concentration of vitamin B12 in these injectable formulations is between 0.01 mg/mL and 10 mg/mL.

In another embodiment, an oral solution formulation of B12 contains therapeutically effective amount of vitamin B12, carrageenan, potassium acetate, a sweetener(s), a coloring agent(s), a flavor(s), hydrochloric acid and/or sodium hydroxide to adjust the pH between 4.5 and 7.5, antioxidant, and purified water.

In another embodiment, a nasal spray formulation of B12 containing therapeutically effective amount of vitamin B12, hydroxypropyl methylcellulose, carrageenan, potassium acetate, a humectant, a preservative, a buffer and purified water is prepared. The solution may have higher viscosity compared to the injectable vitamin B12 formulation and it may be in the form of flowable gel.

In another embodiment, a nasal spray formulation of B12 containing therapeutically effective amount of vitamin B12, carrageenan, potassium acetate, a humectant, a preservative, a buffer and purified water is prepared. The concentration of vitamin B12 in the nasal spray formulation is between 0.01 mg/mL and 10 mg/mL.

Vitamin B12 is available in various forms. For the purpose of this invention, vitamin B12 can be methyl cobalamin, cyanocobalamin, hydroxocobalamin and adenosylcobalamin. The following are the key words used and the definition of various terms applicable to this patent application.

### DETAILED DESCRIPTION

### Definition of Terms Used

A drug is “a chemical substance used in the treatment, cure, prevention, or diagnosis of disease or used to otherwise enhance physical or mental well-being. Medicament is a medicine, or a substance used in a therapy. In this patent application, the terms “drug” and “medicament” have been used interchangeably. In this patent, vitamin B12 is considered as a drug.

A “medicament” is an agent that promotes recovery from an ailment or an injury. Similar words to medicament are medicine, drugs, therapeutic agent and an active moiety.

The “therapeutically effective amount” is the amount of pharmaceutical or nutraceutical substance that treats, totally or partially, a disease state or alleviates one or more symptoms of the condition.

The term “comprising” or “comprises” is synonymous with “including”, “containing” and “characterized by”, is inclusive or open-ended.

There are various sizes of hard capsule shells available ranging from 000 to 5, the most commonly used are sizes 0 and 1. The fill weight of granules with a density of 0.7 g/mL is 475 mg and 350 mg for size 0 and 1 capsules, respectively. Size 000 capsule can contain 960 mg of core material loaded with medicament. The average weights of size 1 and 0 empty hard gelatin capsule shell are 75 mg and 98 mg, respectively. These weights can vary, but overall the weights are low. The drug loading in the capsule shell composition can affect the properties of the capsule shell; and thus, the amount of drug loaded in the capsule shell is limited. In general, only potent drugs can be loaded in capsule shells. Also, one skilled in the art will determine the stability of the drug in the capsule shell composition during manufacture and storage. It is important to establish the desired release rate of medicament from the capsule shell under pre-determined conditions. The polymer for the capsule shell and the drugs to be incorporated in the capsule shell matrix need to be selected judiciously.

Bioavailability of a drug constitutes of two features—the rate and the extent of absorption. For drugs with narrow therapeutic indices, it is critical to maintain appropriate drug levels in the blood or tissues. For potent drugs, one must avoid dumping of drugs in a short period from the delivery device into the gastro-intestinal tract so that one can avoid erratic blood levels of the medicament. In the current application, it is desired to allow the absorption of significant amount of vitamin B12 from buccal cavity. It is important to make sure that B12 is released in the mouth cavity in sufficient quantity and over a long time.

Pharmaceutical capsule dosage forms are widely used in delivering drugs. Composition is the combination of parts or elements and it is the way in which something is put together. The composition of the capsule shell consists of a list of ingredients incorporated and their respective quantities. The main two types of capsules are—hard-shell capsules and soft-shell capsules. The term “hard” for a substance or matter is defined as a solid, firm substance or matter which is resistant to pressure and with endurance. Polymer is defined as a large molecule, or macromolecule, composed of many repeated subunits. A system is defined as a set of connected things or parts forming a complex whole. A polymeric system means a system in which one of the major components (in terms of quantity and functionality) is a polymer. The polymeric system may have other components such as a plasticizer, color, lubricant, cross-linking agent etc. A structure is the arrangement of and relations between the parts or elements of something complex. A structure has a form, shape, composition, formation or a constitution. A hard polymer structure is defined as a structure with polymer as a major component. Hard capsule polymeric structure indicates a structure with a shape of capsule, which is solid and does not change with time, external pressure and it has a polymer or a mixture of polymers as the major component. A hard capsule has a body and cap. It is the body in the core of which holds the contents of the dosage form (“capsule core formulation or capsule core material”). The cap is placed on the body and pressed to stay locked. In contrast, the soft polymer capsule structure is soft in nature and can change the shape by some pressure. Soft capsule shells have higher amounts of water/moisture compared to the hard-capsule shell. The manufacturing processes for the hard and soft capsule shells are different. As mentioned earlier, in the case of hard capsule, body and caps are prepared separately and are put together to form the entire capsule. For HPMC size 0 capsules, the average total weight of 10 capsules was observed to be 97.2 mg. The average weights of caps and body were found to be 38.0 mg and 59.2 mg, respectively. It shows that caps are smaller than the body of a capsule.

A “capsule shell” is referred to as a film-forming composition used to encapsulate an active moiety in a capsule dosage form. For this patent, in one of the formulations, the capsule shell-forming composition also contains a drug or drugs.

The “empty, hard-shell capsule” as name suggests is hard, durable and smooth capsule. It retains its shape and it is dry in nature. As evident from the word “empty”, there is nothing inside core portion of the capsule shell when supplied by the capsule manufacturing company. Currently such “empty, hard-shell capsule” compositions available in the market do not contain any drug(s). The “capsule core material” may be prepared from a wide variety of materials including, but not limited to, sweeteners, flavors, medicaments, coloring agents, dispersing agents, lubricants and glidants etc. The “capsule core material” can be in the form of but not limited to powder, granules, beads, pellets, microspheres, micro-capsules, mini-tablets and mini-capsules. Someone may argue that the body is type of a core. However, people working in the field of pharmaceutical formulations will not confuse the “body” of the capsule shell with the material within the core of the capsule.

The “drug-loaded empty, hard-shells capsules” means the empty, hard capsule shells in which a drug is loaded in the shell composition. The term “loaded” means the drug is either physically dispersed or molecularly dissolved in the shell composition or chemically bound to the polymeric material incorporated in the capsule shell-forming composition. The term “molecularly dissolved” in the above sentence means, the drug(s) is dissolved molecularly in the capsule shell-forming composition. Undissolved particles less than 200 nm particle size are not visible to the necked eyes and can be considered “dissolved”. The term “chemically bound to the polymeric material” in the above sentence means, the drug is ion-paired or complexed or covalently bound to the gelatin or polymer used to form the capsule shell. These drug-loaded empty hard-shell capsules are prepared by incorporating drug in the capsule shell-forming composition, which is then converted to a capsule shape (body and cap). In this patent application, vitamin B12 has sufficient water solubility and it will be in a molecularly dissolved state in the capsule forming solution.

The term “essential” means absolutely necessary. The hard-shell capsule must have some essential physical and chemical properties. Property of a substance is a characteristic, which helps to identify, describe, define or quantify the substance. The capsule shell is thin. Empty capsules shells are packed in big plastic bags and should not break during transportation and storage. After filling drugs, the capsules are stored in small bottles or in the blister packs. Capsule shells should not break during transportation and storage in these primary packaging systems. For that, they should have sufficient elastic stiffness, tensile strength and should not be brittle. The drug incorporated in the shell matrix should not affect the essential physical and chemical properties. In the current application, the shell matrix is expected to dissolve in the mouth cavity.

Each capsule has two parts—a body and a cap. Commonly used primary ingredients in the composition of the capsule body or the cap are gelatin and hydroxypropyl methyl cellulose. As mentioned in the earlier section, apart from gelatin, several new types of polymeric substances have been used to manufacture hard capsule shells. These are—cellulosic compounds, acrylates, starch ethers, polyolefins, pullulans, and carrageenan. Apart from the main constituent of the capsule shell, gelatin or polymer, the shell may also contain other excipients such as plasticizers (e.g., polyethylene glycol, sorbitol, glycerol), stabilizers (antimicrobial and antioxidants), colorants (FD&C colors, titanium dioxide, natural dyes including riboflavin, carotenes, turmeric and caramel) and sequestering agents (citric acid, sodium citrate, ethylenediaminetetraacetic acid).

The rate of capsule dissolution in a desired media governs the release of medicaments residing in the capsule shell matrix or in the core of the capsule. The rate of release may be altered using selected combination of polymers in the capsule composition or by a cross-linking of gelatin or polymer used in the capsule shell formulation.

The present invention proposes a method to stabilize vitamin B12 in liquid formulations. The liquid formulation approach is utilized to develop four types of dosage forms—1. A liquid formulation, which is used to manufacture capsule shell matrix (cap and body), 2. Injectable formulation, 3. Nasal spray and 4. Oral liquid.

There are two key phrases—“capsule shell-forming composition” and “capsule-core material/formulation”.

Capsule shell-forming composition described herein includes a composition suitable for forming empty hard-shell capsules. In some embodiments, vitamin B12 is added to the capsule shell-forming composition. The capsule shell-forming compositions may be different for capsule cap and capsule body. Vitamin B12 may reside only in the composition of the cap or only in the composition of the body or in the compositions of both cap and the body.

An “excipient” as used herein is more or less an inert substance added as diluents or vehicles or to give form or consistency or properties. Excipients may also act as a preservative. A “preservative” is understood herein to mean certain embodiments which are substances added to inhibit chemical change or microbial growth. When the preservative inhibits microbial growth, such preservatives may include, but are not limited to sodium benzoate, methylparaben, propyl gallate, sorbic acid, chlorobutanol, dihydroacetic acid, monothioglycerol, potassium benzoate, propylparaben, benzoic acid, benzalkonium chloride, benzethonium chloride, benzyl alcohol, butylparaben, cetylpyridinium chloride, ethylenediamine, ethylparaben, thimerosal, and potassium sorbate.

The formulations claimed in this patent are aqueous in nature. i.e., water is used as the solvent. In the preparation of injectable formulations, water for injection is used. Water for injection is water of extra high quality without significant contamination. It is assumed to be sterile. A sterile version is used for making solutions that will be given by injection. Purified water is used to make the formulation used to make capsules or a nasal spray formulation or oral formulation. To prepare purified water, multiple impurities are removed, including ions. Deionized water contains very low concentration of ions but may contain impurities like bacteria or particulates which are not present in purified water. No water is considered purified if it is no deionized.

In certain embodiments, the capsule shell-forming composition may also contain a flavoring agent. A “flavoring agent” is understood herein to mean certain embodiments which are substances that alter the flavor of the composition during oral consumption. A type of “flavoring agent” would be a sweetener. Preferred sweeteners can be natural or artificial. Flavoring agents can be chosen from a group commonly used in the pharmaceutical dosage forms. A “sweetener” is a substance that provides a sweetening effect. Sweetener is one especially other than sugar.

In certain embodiments, vitamin B12 liquid composition may also contain a buffering agent. A “buffering agent” is a weak acid or a weak base or a mixture of two, which helps to prevent fluctuations in the pH of the medium when exposed to an acidic or basic environment. Some of the examples of “buffering agents” may be, but not limited to, phosphate, citrate, borate, phthalate and acetate.

A humectant is a substance which retains moisture in the formulation or keep thing moist. Humectants are found in moisturizers. Humectants are used in some of the vitamin B12 formulation and are selected from the group consisting of sorbitol, propylene glycol, polyethylene glycol, glycerin and mixtures thereof.

A combination drug therapy has been gaining a lot of importance in recent times. The combination therapy in medicine involves administration of two or more therapeutically active ingredients. The active ingredient may be administered in a single dosage form or each active ingredient may be administered separately. The reasons of a combination therapy could be multiple—synergistic effects of drugs, reduction of side effect of the primary drug by an adjunct drug, avoidance of taking multiple tablets/capsules per day thereby savings on co-payment for different medicines and assurance of patient compliance to drug therapies. In some embodiments of the combination drug therapy, the active ingredients are administered in a single dosage form. In those instances, it is important to show that different drugs combined in the same dosage form are stable during storage of the dosage form and should not interact physically or chemically with other drugs or excipients to produce degradation products. Also, each drug should show the desired release rate from the dosage form to get absorbed in sufficient quantities upon oral administration or release the drug to surrounding environment in case of other delivery routes.

The polymers that can be used in making the present hard capsule shells can be divided into the following groups: 1) Cellulose- or cellulose derived-based material, which include, but are not limited to, cellulose, cellulose ether, methyl cellulose, hydroxypropyl methyl cellulose, hydroxyproyl cellulose, hydroxyethyl cellulose, carboxymethyl cellulose, cellulose acetate phthalate. 2) starch-based compounds, which include, but not limited to hydroxyethyl starch, hydroxypropyl starch, hydroxyethyl methyl starch, 3) carrageenans—kappa, lambda and iota, 4) Acrylate derivatives which include, but not limited to, polyacrylate, polymethylacrylate, poly(acrylate-methylacrylate), poly(methylacrylate-methyl methacrylate), 5) polyolefins which include, but limited to, polyvinyl chloride, polyvinyl alcohol, and polyvinyl acetate and 6) pullulan (a polysaccharide polymer consisting of maltotriose units).

An artificial sweetener is a synthetic compound which can be a sugar substitute as it provides a sweet taste like that of sugar. Some of the commonly used artificial sweeteners are—Aspartame. Sodium saccharin, sucralose, stevia etc. A flavoring agent is a compound which imparts a pleasant flavor to the product such as a capsule. There are a variety of food flavors used in the pharmaceutical industry. They can be used alone or in combination with others.

Synergism is an interaction or cooperation of two or more substances to produce a combined effect greater than the sum of their individual effects.

As the name suggests, an injectable formulation is the one which can be injected. There are several types of injectables—subcutaneous injection is injected underneath the skin; intramuscular injection is delivered into the muscle. As the name suggests, nasal sprays or nasal gels are formulations are delivered to the nasal cavity. Oral liquid is a solution, syrup, suspension, elixir and a concentrate which is fed into the mouth cavity to be swallowed.

An antioxidant is a substance that inhibits oxidation of products. Nitrous oxide is known to inactivate cobalamin form of vitamin B12. Vitamin B12 comprises a cobalt (Co)-containing tetrapyrrole ring with a variable upper axial ligand: methyl-, adenosyl-, hydroxo-, or cyano-groups. Dimethylbenzimidazole is bound to one pyrrole and coordinates to cobalt as the lower axial ligand. The cobalt atom can exist in three oxidative states in cobalamin. When Co is coordinated with pyrrole ring alone, it is known as cob(I)alamin state. When Co binds with pyrrole ring, and upper and lower ligands, it is known as cob(III)alamin state. Removal of one axial ligand leaves cobalt in the cob(II)alamin state. Dereven'kov et al. (2013) found that Co(I) species was oxidized by thiosulfate, sulfite and dithionite to Co(II) forms: oxidation by excess thiosulfate lead to penta-coordinate complexes and oxidation by excess sulfite or dithionite lead to hexa-coordinate complexes. On the same lines, cyanocobalamin (B12) and hydroxocobalamin (B12b) were found to degrade in the presence of ascorbic acid (AH2) at pH 1.0-8.0, with a maximum rate at pH 5.0 (Ahmad et al., 2014). The degradation of B12 and B12b involves the reduction of Co+3 to Co2+ in the corrin ring by AH2, to form corrin macrocyclic ring cleavage products. Patel K. et al. described a nasal composition of vitamin B12 (EP2632430). They sodium glycocholate as the permeation enhancer and the in vivo nasal absorption was shown to be significantly more than Nascobal. They prepared their formulation under nitrogen flushing under sodium vapor lamp. The dissolved oxygen levels were kept at about 1 to 1.5 ppm. Based on this information, it seems that antioxidants should not in added to the formulations and the process should be conducted under nitrogen overlay. Nitrogen overlay means, a continuous flow of nitrogen is used to maintain low oxygen concentration within a tank headspace. At small-scale, a glove-box can be used in which the air is replace by nitrogen.

Vitamin B12 is known to be light sensitive and therefore, during the manufacturing process, vitamin B12 products should be protected from light to prevent light degradation. Photodegradation occurs normally due to absorption of short wavelength light between 300 nm and 500 nm. In this patents, long wavelength light is defined as one with a wavelength more than 500 nm. Some of these lights are—Green: 520-555 nm, Yellow: 585-600 nm, Amber: 600-615 nm, Orange-red: 625-640 nm and Red: 640-700 nm etc.

The release of drug, which is embedded in the capsule shell composition, may be controlled by two mechanisms—diffusion through the shell matrix or by dissolution of the hard-capsule shell to release the drug.

The term “slow-release ODC” is different from rapid or fast-release ODC. “Rapid/Fast disintegration/dissolution” as used herein should be understood to encompass disintegration/dissolution of at least 60% of the core composition of the ODC, typically 90% and more typically 100% of the core composition in an aqueous medium or in saliva (in the oral cavity) within 30 seconds and at times, even within 5 to 15 second. Rapid/fast ODC is meant to release the drug very fast in 10 seconds to 20 seconds. The term “slow release” refers as dissolution of at least 60% of the composition of the invention, typically 90% and more typically 100% of the composition in an aqueous medium or in saliva (in the oral cavity) in more than 3 minutes and within 60 minutes. In this case, the dissolution test of ODC is conducted in the mouth cavity and not in the USP dissolution apparatus, which uses 900 mL volume per vessel. The volume of 900 mL in the USP is not representative of volume in the mouth cavity. Specifically, the ODC, in this case, is placed in the buccal cavity. Buccal cavity is that part of the mouth bounded anteriorly and laterally by the lips and the cheeks, posteriorly and medially by the teeth and/or gums, and above and below by the reflections of the mucosa from the lips and cheeks to the gums. It can be also called the vestibule of mouth. This study teaches a method to administration of ODC. The ODC can be kept in the middle of the mouth cavity. The ODC will move freely and can be sucked on by the tongue. The study teaches to place the ODC on the side of the mouth between the cheek and the gums. The ODC is left alone to dissolve slowly and not move in the mouth cavity from right to left or left to right. The ODC may be rotated in its place by tongue, if necessary.

The advantages of slow-release ODCs are—it has a high resident time in the mouth cavity making the drug available to be absorbed locally, it reduces hepatic first pass effect by allowing absorption of drug from the mouth cavity, dose accuracy in comparison to oral liquids, no need of water or a spoon for administration and lowering of T-max, which is time to achieve the maximum drug concentration in the blood. The hepatic first pass effect or the first pass metabolism is a phenomenon of drug metabolism in the liver whereby the concentration of a drug is greatly reduced before it reaches the systemic circulation. Thus, for drugs which can be absorbed from the buccal cavity, we can achieve lower Tmax and higher AUC or we can reduce the dose of the drug to achieve same pharmacological effect.

Synergy is an interaction or cooperation of two or more substances, or other agents to produce a combined effect greater than the sum of their separate effects. We observed a synergistic stabilizing effect during the stability studies of vitamin B12 in liquid formulations.

Stability of Vitamin B12 in an Aqueous Solution

Vitamin B12 is light and thermal sensitive material, especially in the liquid formulations. During the manufacture of capsules, the polymer or gelatin is a first dissolved in water to which other excipients are added. The solution is maintained at about 60° to 80° C. This solution is then used to manufacture capsule caps and bodies. It takes about 8 to 10 hours. Thus, the key aspect of this patent is to prove the stability of vitamin B12 in the aqueous solution containing a polymer or a gelatin. HPLC assay was developed in order to perform stability studies.

A typical polymeric capsule contains HPMC, carrageenan, potassium acetate and water. Five HPMC capsules were dissolved in 10 mL water to which vitamin B12 was added. The solution was kept at 40′, 60° and 80° C. for 24 hours and the samples were analyzed for B12 contents. Table 3 lists the % assay values. Vitamin B12 in water was used as the control. Clearly, the solutions were observed to be stable at 40° C. temperatures, degraded more at higher temperatures especially at 80° C. after 24 hours of storage. Vitamin B12 in the presence of capsule component showed better stability. Sample without the capsule components showed 86.2% assay value after autoclaving and in the sample with the capsule, the same value was 98.0%. It clearly showed that HPMC capsule components helped to stabilize vitamin B12 in an aqueous solution.

TABLE 3
Effect of Temperature on Vitamin B12

| | % Assay values | | | | | |
|---|---|---|---|---|---|---|
| | after autoclaving | | % Assay | % Assay values | | |
| | Before | After | values | after 24 Hrs | | |
| | Autoclaving | autoclaving | Zero time | 40° C. | 60° C. | 80° C. |
| S-1 | 101.4 | 86.2 | 101.4 | 99.5 | 98.8 | 90.4 |
| S-2 | 104.4 | 98.0 | 104.4 | 100.7 | 99.4 | 99.2 |

Note:
S-1: Cyanocobalamin with water and
S-2: Cyanocobalamin with HPMC capsule in water

In another experiment, the stability of vitamin B12 was examined in different concentrations of HPMC. Table 4 lists the % assay recovery values of the samples analyzed. The samples were stored at different temperatures for 24 hours. Samples were also autoclaved at 121° C. for 15 minutes. From Table 4, it can be seen that all HPMC samples showed slightly better stability as compared to as is or No HPMC sample at 80° C. Evaluation of the same data also suggested that there was no effect of different concentrations of HPMC on the stability of B12. Comparison of data from Table 4 with the data from Table 3, we could conclude that % assay values of B12 in samples containing only HPMC were not as good as the values for Sample 2 in Table 3. Sample 2 contained HPMC capsule components dissolved in water with vitamin B12. Something else in capsule composition helped to stabilize B12 in the sample. This must be either carrageenan or potassium acetate present in the HPMC capsule.

TABLE 4
Effect of HPMC on the stability of Vitamin B12, Values compared
to values at 40° C. or values prior to autoclaving.

| | Effect of different | | | Effect of | |
|---|---|---|---|---|---|
| | temperatures, 24 hrs | | | Autoclaving | |
| B12Sample | 40° C. | 60° C. | 80° C. | Before | After |
| No HPMC | 100 | 96.8 | 89.7 | 100 | 87.3 |
| 0.25% HPMC | 100 | 101.6 | 95.5 | 100 | 90.2 |
| 1% HPMC | 100 | 100.3 | 93.6 | 100 | 87.1 |
| 2% HPMC | 100 | 99.2 | 93.0 | 100 | 88.2 |

In yet another experiment, the stability of vitamin B12 was examined in different concentrations of Carrageenan. In 10 mL water, different amounts of carrageenan were added. The amounts of potassium acetate were half the quantities of carrageenan. All the samples contained vitamin B12. In one sample, 5 HPMC capsules were dissolved in 10 mL water to which vitamin B12 was dissolved. Table 5 lists the % assay recovery values. The samples were stored at different temperatures for 24 hours. Samples were also autoclaved at 121° C. for 15 minutes. Stability data from Table 5 suggest that carrageenan helped to stabilize vitamin B12 in samples stored under various conditions. Vitamin B12 in sample containing no carrageenan showed 62.5% assay value at 80° C. The assay values increased in samples upon increasing the amount of carrageenan. Sample containing 10 mg carrageenan and 5 mg potassium acetate showed 93.6% assay value. A similar value was observed in the sample in which 5 HPMC capsules were dissolved in 10 mL water. Vitamin B12 formulation with and without carrageenan which was autoclaved also showed significant difference in terms of assay values. Sample of vitamin B12 without carrageenan showed % assay value of 81.6% after autoclaving. Sample containing 10 mg carrageenan and 5 mg potassium acetate showed 92.3% assay value after autoclaving. An assay value of 98.4% was observed in the sample in which 5 HPMC capsules were dissolved in 10 mL water. Based on these two experiments, HPMC and carrageenan helped to stabilize vitamin B12 in an aqueous solution, carrageenan showing better stabilization compared to HPMC. HPMC capsule composition contained HPMC, carrageenan and potassium acetate and it stabilized vitamin B12 most.

TABLE 5
Effect of Carrageenan on the stability
of Vitamin B12, % assy values.

| | Effect of different | | | Effect of | |
|---|---|---|---|---|---|
| | temperatures, 24 hrs | | | Autoclaving | |
| | (% Assay values) | | | (% Assay values) | |
| B12 Sample | 40° C. | 60° C. | 80° C. | Before | After |
| No | 101.9 | 99.6 | 62.5 | 100 | 81.6 |
| Carrageenan | | | | | |
| 1 mg C + | 94.9 | 94.2 | 68.2 | 100 | 81.5 |
| 0.5 mg KA | | | | | |
| 3 mg C + | 97.7 | 103 | 68.9 | 100 | 87.8 |
| 1.5 mg KA | | | | | |
| 5 mg C + | 99.9 | 104 | 70.6 | 100 | 90.6 |
| 2.5 mg KA | | | | | |
| 10 mg C + | 96.47 | 99.5 | 93.6 | 100 | 92.3 |
| 5 mg KA | | | | | |
| 5 HPMC | 97.76 | 99.8 | 93.3 | 100 | 98.4 |
| capsules in | | | | | |
| 10 mL water | | | | | |

C = carrageenan,
KA = potassium acetate

In yet another experiment, the effect of the combination of carrageenan and HPMC on the stability of vitamin B12 was examined. Table 6 lists the % assay values. The samples were stored at different temperatures for 24 hours. Stability data from Table 6, it seems carrageenan and HPMC together have synergistic effect that helped to stabilize vitamin B12 in various conditions. The % assay values at 40° C., 60° C. and 80° C. after 24 hours of storage were very similar. Normally, the % assay values were observed to be lower at 80° C. Assay values after autoclaving were compared with assay values prior to autoclaving. The samples showed stabilization in the presence of carrageenan, HPMC and potassium acetate. The pH values of these two samples were 6.4 and 6.22. The viscosity values were measured using a Canon viscometer. The solutions were free flowing and viscosity values were less than 10 cps.

TABLE 6
Effect of HPMC + Carrageenan on the stability
of Vitamin B12, % peak area for Vitamin B12 peak,

| | Effect of different | | | Effect of | | | |
|---|---|---|---|---|---|---|---|
| | temperatures, 24 hrs | | | Autoclaving | | | Viscosity, |
| Sample | 40° C. | 60° C. | 80° C. | Before | After | pH | cps |
| 1 mg/ml C + 5 mg/ml | 96.5 | 96.1 | 96.7 | 100 | 100.9 | 6.40 | 5.72 |
| HPMC + 0.5 mg/ml K | | | | | | | |
| acetate | | | | | | | |
| 1 mg/ml C + 2.5 mg/ml | 95.5 | 95.8 | 95.1 | 100 | 97.7 | 6.22 | 4.41 |
| HPMC + 0.5 mg/ml K | | | | | | | |
| acetate | | | | | | | |

C = carrageenan

In yet another experiment effect of pH on the stability of solutions of vitamin B12 was studied. It was found that the solutions of vitamin B12 were more stable at pHs between pH 4 and 6.5.

Examples of Various Vitamin B12 Liquid Formulations

With the following examples, one skilled in the art, can understand and use the present invention. Many formulations can be developed using the stabilized B12 composition.

### Example 1

Vitamin B12 in the Capsule Shell Matrix—Delivered as an Empty Capsule

Almost 30% of people taking metformin face vitamin B12 deficiency. Vitamin B12 is dissolved in the HPMC or gelatin capsule shell-forming composition. Empty capsule shells are prepared using this B12-loaded shell-forming composition. One can administer these capsules to patients without filling anything into the capsule shell.

TABLE 7
Composition of empty Hard capsule containing
vitamin B12, size 00 capsule

| Ingredient | Amount | |
|---|---|---|
| Cyanocobalamin | 2 | mg |
| Hydroxypropyl methylcellulose | 114 | mg |
| Aspartame | 2 | mg |
| Glycerin | 1 | mg |

HPMC is dissolved in purified water at 80° C. along with glycerin, protected from light and under nitrogen over-lay. Vitamin B12 is added to the polymer solution once the polymer dissolved completely. The cap and body of the empty capsules are prepared by the pin-dip method. Both the cap and body of the capsule shell contain vitamin B12 and the empty capsule is administered as is. The capsule is kept between gum and cheek for a slow release of Vitamin B12.

### Example 2

Vitamin 12 in the capsule shell matrix—capsule core filled with inert material

TABLE 8
Composition of empty Hard capsule containing vitamin
B12 and then filled with an inert material

| | |
|---|---|
| Ingredient | Amount |

Composition of empty hard capsule shell, size 00

| | | |
|---|---|---|
| Cyanocobalamin | 2 | mg |
| Hydroxypropyl methylcellulose | 114 | mg |
| Aspartame | 2 | mg |
| Glycerin | 1 | mg |

Composition of material filled in the capsule
shell matrix containing Vitamin B12

| | | |
|---|---|---|
| Cocoa powder | 130 | mg |
| Granular sugar | 530 | mg |

The empty hard-shell capsule loaded with vitamin B12 is prepared by the pin-dip method. A mixture of cocoa powder and granular sugar is prepared separately and filled in the empty hard-shell capsules containing vitamin B12 in the capsule shell matrix. The capsule is kept between gum and cheek for a slow release of Vitamin B12. The outer capsule shell dissolves releasing vitamin B12 to be absorbed in the mouth cavity. The mixture of cocoa and sugar is released slowly in the mouth cavity.

### Example 3

Vitamin B12 in the Capsule Shell Matrix—Capsule Core Filled with Metformin

Metformin has a bitter taste and it is important to mask the taste in this formulation.

TABLE 9
Composition of empty Hard capsule containing vitamin
B12 and then filled with metformin granules

| | |
|---|---|
| Ingredient | Amount |

Composition of empty hard capsule shell, size 00

| | | |
|---|---|---|
| Cyanocobalamin | 2 | mg |
| Hydroxypropyl methylcellulose | 114 | mg |
| Aspartame | 2 | mg |
| Glycerin | 1 | mg |

Composition of material to be filled in the
capsule shell matrix containing Vitamin B12

| | | |
|---|---|---|
| Metformin | 500 | mg |
| Sucralose | 0.5 | mg |
| Peppermint flavor | 2 | mg |
| Povidone | 75 | mg |
| Magnesium stearate | 20 | mg |

| | |
|---|---|
| Cellulose Acetate Phthalate | Quantity sufficient to coat |

The empty hard-shell capsule loaded with vitamin B12 is prepared by the pin-dip method. A mixture of metformin, povidone is prepared separately and granulated with water. The granules are coated with cellulose acetate phthalate solution, dried and magnesium stearate is added. The granules are filled in the empty hard-shell capsules containing vitamin B12 in the matrix. As the granules are small, the patient can swallow them easily with saliva.

### Example 4

Vitamin B12 in the capsule shell matrix—capsule shell filled with carbamazepine.

TABLE 10
Composition of empty Hard capsule containing vitamin
B12 and then filled with carbamazepine granules

| | |
|---|---|
| Ingredient | Amount |

Composition of empty hard capsule shell, size 00

| | | |
|---|---|---|
| Cyanocobalamin | 2 | mg |
| Hydroxypropyl methylcellulose | 114 | mg |
| Glycerin | 1 | mg |

Composition of material to be filled in the
capsule shell matrix containing Vitamin B12

| | | |
|---|---|---|
| Carbamazepine | 200 | mg |
| Citric acid | 75 | mg |
| Citrous flavor | 25 | mg |
| Corn Starch | 100 | mg |
| Sucrose | 250 | mg |
| Magnesium stearate | 20 | mg |

The empty hard-shell capsule loaded with vitamin B12 is prepared by the pin-dip method. A mixture containing carbamazepine, citric acid, citrous flavor, corn starch and sucrose is prepared separately and granulated with water. Magnesium stearate is added to the granules. The granules are filled in the empty hard-shell capsules containing vitamin B12 in the matrix. The capsule are swallowed with water. Drugs are released in the GI tract.

### Example 5

Example of Vitamin B12 Injection

TABLE 11
Composition of Vitamin B12 Injection

| Ingredient | Amount per mL | |
|---|---|---|
| Vitamin B12 | 1 | mg |
| Hydroxypropyl methylcellulose | 2.5 | mg |
| Carrageenan | 1 | mg |
| Potassium acetate | 0.5 | mg |
| Sodium chloride | 9 | mg |

| | |
|---|---|
| HCl or NaOH to adjust the pH | Between 4.5 and 7.5 |
| Water for Injection | q.s. to 1 mL |

### Example 6

Example of Vitamin B12 Oral Solution

TABLE 12
Composition of vitamin B12 oral solution

| Ingredient | Amount per 5 mL | |
|---|---|---|
| Vitamin B12 | 1 | mg |
| Hydroxypropyl methylcellulose | 15 | mg |
| Carrageenan | 5 | mg |
| Potassium Acetate | 2.5 | mg |
| Anhydrous citric acid | 1 | mg |
| Sodium citrate | 15 | mg |
| Edetate disodium | 5 | mg |
| FD&C red # 40 | 2 | mg |
| Glycerin | 1 | gm |
| Sodium Benzoate | 15 | mg |
| Sorbitol solution | 1 | gm |
| Strawberry flavor | 25 | mg |
| Sucralose | 10 | mg |

| | |
|---|---|
| Purified water | q.s. to 5 mL |

### Example 7

Example of Vitamin B12 Nasal Spray

TABLE 13
Composition of Vitamin B12 Nasal spray

| Ingredient | Amount per mL | |
|---|---|---|
| Vitamin B12 | 5 | mg |
| Hydroxypropyl methylcellulose | 2.5 | mg |
| Carrageenan | 1 | mg |
| Potassium acetate | 0.5 | mg |
| Sodium citrate | 1.85 | mg |
| Citric acid | 0.70 | mg |
| Glycerin | 10 | mg |
| Benzalkonium chloride | 1 | mg |

| | |
|---|---|
| HCl or NaOH to adjust the pH | Between 4.5 and 6.5 |
| Purified water | q.s. to 1 mL |

### Example 8

Example of Methotrexate with Vitamin B12

TABLE 14
Composition of methotrexate and vitamin B12 oral solution

| Ingredient | Amount per 5 mL | |
|---|---|---|
| Methotrexate disodium | 10.95 | mg |
| Vitamin B12 | 1 | mg |
| Hydroxypropyl methylcellulose | 15 | mg |
| Carrageenan | 5 | mg |
| Potassium acetate | 2.5 | mg |
| Sodium methyl parahydroxybenzoate | 10 | mg |
| Sodium ethyl parahydroxybenzoate | 5 | mg |
| Sodium Citrate | 15 | mg$ |
| Citric acid monohydrate | 1 | mg$ |
| Sucralose | 10 | mg |
| Raspberry flavor | 25 | mg |

| | |
|---|---|
| Purified water | q.s. to 5 mL |

### Example 9

Example of Ranitidine Hydrochloride Oral Solution with Vitamin B12

TABLE 15
Composition of ranitidine hydrochloride
oral soltion with vitamin B12

| Ingredient | Amount per mL | |
|---|---|---|
| Ranitidine (as hydrochloride) | 25 | mg |
| Vitamin B12 | 1 | mg |
| Hydroxypropyl methylcellulose | 2.5 | mg |
| Carrageenan | 1 | mg |
| Potassium acetate | 0.5 | mg |
| Monobasic potassium phosphate | 0.96 | mg |
| Dibasic sodium phosphate | 2.4 | mg |
| Sodium saccharin | 5 | mg |

| | |
|---|---|
| Purified water | q.s. to 1 mL |

From the foregoing, it is clear that this patent application opens up several possibilities using the vitamin B12-loaded liquid formulations. One of the formulations is used to prepare empty capsule shells loaded with vitamin B12 in the capsule shell matrix. These capsules can be used as is for the administration of vitamin B12 or can be filled with inert material, Vitamin B12 capsules can be used as a carrier of different medicaments in the core. Carrageenan, HPMC and potassium acetate are added to injectable, nasal and oral liquid formulations as they stabilized vitamin B12. While specific embodiments have been presented here, various modifications can be made, and the invention is not limited to the examples shown in this patent application. There are mainly three independent claims—one for a solution used to prepared B12 loaded capsule shell matrix, second one for vitamin B12 injectable formulation and there third one is the vitamin B12 nasal formulation.

Overall, an aqueous solution of vitamin B12 can be stabilized by the synergistic effect of HPMC, carrageenan and potassium acetate. While the invention has been described in connection with specific embodiments thereof, it will be understood that it is capable further modifications apparent to those skilled in the art. Such changes and modifications can be made by those skilled in the art without departing from the spirit and scope of the present subject matter and without diminishing its intended advantages.

### REFERENCES

1. Ahmad I., Qadeer K., Zahid S., Sheraz M. A., Ismail T., Hussain W., and Ansari I. A.; Effect of ascorbic acid on the degradation of cyanocobalamin and hydroxocobalamin in aqueous solution: a kinetic study, AAPS PharmSciTech 15: 1324-1333 (2014).
2. Aroda, V. R., Edelstein S. L., Goldberg R. B., Knowler W. C., Marcovina S. M., Orchard T. J., Bray G. A., Schade D. S., Temprosa M. G., White N. H. and Crandall J. P., Long-term Metformin Use and Vitamin B12 Deficiency in the Diabetes prevention program Outcomes study, J. Clin. Endocrinol. Metab., 101: 1754-1761 (2016).
3. Aslan K, Bozdemir H., Unsal C. and Guvenc B; The effect of antiepileptic drugs on vitamin B12 metabolism; Int. J. Lab Hematol. 30: 26-35 (2008).
4. Canadian Nutrient File 2015” www.hc-sc.gc.ca/fn-an/nutrition/fiche-nutri-data/index-eng.php
5. Castelli, M. C., Friedman, K., Sherry, J., Brazzillo, K, Genoble, L, Bhargava, P., and Riley, M. G. I., Comparing the efficacy and tolerability of a new daily oral Vitamin B12 formulation and intermittent intramuscular vitamin B12 in normalizing low cobalamin levels: A randomized, open-label, parallel-group study, Clinical Thera. 33: 358-371 (2011).
6. Chinna R. P., Chaitanya, K. S. C. and Madhusudan Rao, Y. A review of bioadhesive buccal drug delivery systems: current status of formulation and evaluation methods; DARU J. Pharm. Sci. 19: 385-403 (2011).
7. Cruz, A. and Pristupa, Z, Vitamin B12-containing compositions and methods of use, US patent #2008/0039422 (2008).
8. Dereven'kov, L. A., Salnikov, D. S., Makarov, S. V., Boss, G. R. and Koifman, 0.1. Kinetics and mechanism of oxidation of super-reduced cobalamin and cobinamide species by thiosulfate, sulfite and dithionite, Dalton Trans. 42(43): 15307-15316 (2013).
9. Goldberg, M. and Arbit, E. Edible strips, WO/2013/028333 (2012).
10. Gutierrez, P., and Beer, C, Dietary supplement for improving brain health, US patent #2018/0085310 (2018).
11. Heep, I., Stabilization of vitamin B12, U.S. patent 2011/0065665 (2011).
12. Heep, I and Taterra, H. R., Stabilization of Vitamin B12, U.S. Pat. No. 9,089,582 (2015).
13. Huang H. L., Zhou, H., Wang, N and Yu, C. Y.; Effects of antiepileptic drugs on the serum folate and vitamin B12 in various epileptic patients; Biomedical reports 5: 413-416 (2016).
14. Jain, K. D., Jayanthi, C. Singh, M., Roopa G. and Joshi, K. H., Comparison study of vitamin B12 for its efficacy and bioavailability of various formulations in the treatment of pernicious anemia, Int. J. Pharmacy and Pharm. Sciences, 7: 6-8, (2015).
15. Joshi, H. N., Physically dispersed, molecularly dissolved and/or chemically bound drug(s) in an empty, hard capsule shell composition, US Patent 20180116970 (2017)
16. Joshi H. N. and Lad, A. N., Method for a slow release of drugs from orally dissolving capsules, US Patent 20180318226 (2018)
17. Kolber, M. R., and Houle, S. K. D., Oral Vitamin B12: a cost-effective alternative. Canadian Family Physician, 60:111-112 (2014).
18. Linnebank, M., Moskau, S., Semmler, A., Widman, G., Stoffel-Wagner, B., Weller, M., and Elger, C. E., Antiepileptic drugs interact with folate and vitamin B12 serum levels, Ann. Neural. 69: 252-259 (2011).
19. McCarty, J., Method of treating vitamin B12 deficiency, US Patent #201610000716 (2016).
20. Nascobal, Package insert, Reference ID:4360245, 3055827 https://www.accessdata.fda.gov/drugsatfda_docs/label/2018/021642s0191bl.pdf
21. Patel K. R., Patel M. R., and Shah P. J.; Nasal composition of vitamin B12, European patent EP2632430 (2018).
22. Rajewski, R. A., and Haslam, J. L.; Rapidly dissolving Pharmaceutical compositions comprising pullulan, U.S. Pat. No. 8,105,625, 2012.
23. Smart, J. D.; Drug delivery using buccal-adhesive systems; Adv. Drug Delivery Rev. 11: 253-270 (1993).
24. Strong, A. P., Haeusler. S., Weatherall, M., Krebs, J., Sublingual vitamin B12 compared to intramuscular injection in patients with type 2 diabetes treated with metformin: a randomized trial, N. Z. Med. J. 129: 67-75 (2016).

What is claimed:

1. A composition of a liquid used for the preparation of a stable slow-release vitamin B12 orally dissolvable empty capsule shell matrix comprising: (i) a therapeutic dose of vitamin B12; (ii) synergistic amounts of a hard shell-forming material, carrageenan and potassium acetate; (iii) purified water; and (iv) pharmaceutical acceptable excipients; wherein the capsules are prepared under long wavelength light and optionally under nitrogen over-lay; wherein the said capsule shell matrix is prepared using a pin-dip method; wherein optionally a drug(s) known to impair absorption of vitamin B12 is filled inside the core of said capsule.

2. A composition of a liquid used for the preparation of a stable slow-release of vitamin B12 orally dissolvable empty capsule shell matrix as in claim 1 wherein vitamin B12 is selected from a group consisting of cyanocobalamin, hydroxocobalamin, adenosylcobalamin, methyl cobalamin, or mixtures thereof.

3. A composition of a liquid used for the preparation of a stable slow-release of vitamin B12 orally dissolvable empty capsule shell matrix as in claim 1 in which the hard-shell forming material is selected from gelatin, hydroxypropyl methylcellulose, hydroxypropyl cellulose, methyl cellulose and mixtures thereof.

4. A composition of a liquid used for the preparation of a stable slow-release of vitamin B12 orally dissolvable empty capsule shell matrix in claim 1, comprising: a body and a cap prepared using hydroxypropyl methylcellulose polymer as the hard shell-forming material.

5. A composition of a liquid used for the preparation of a stable slow-release of vitamin B12 from a stable orally dissolvable empty capsule shell matrix as in claim 1 wherein the therapeutic dose of vitamin B12 ranges from 0.01 to 10 mg per capsule.

6. A composition of a liquid used for the preparation of a stable slow-release of vitamin B12 orally dissolvable empty capsule shell matrix as in claim 1 wherein the carrageenan is selected from iota carrageenan, kappa carrageenan, lambda carrageenan and mixtures thereof.

7. A composition of a liquid used for the preparation of a stable slow-release of vitamin B12 orally dissolvable empty capsule shell matrix as in claim 1 wherein the capsule size is selected from a group consisting of size 5, size 4, size 3, size 2, size 1, size 0, size 0e1, size 00, size 00e1, and size 000.

8. A composition of a liquid used for the preparation of a stable slow-release of vitamin B12 orally dissolvable empty capsule shell matrix as in claim 1 wherein the placebo or drug-loaded composition in the core-fill is: a powder or granules or microspheres or pellets or mini-tablets or an inner capsule or combination of two or more.

9. A composition of a liquid used for the preparation of a stable slow-release of vitamin B12 orally dissolvable empty capsule shell matrix as in claim 1 wherein the drug(s) known to impair absorption of vitamin B12 is selected from a group consisting of alcohol, antibiotics, gastric acid inhibitor, a biguanide, a proton pump inhibitor, H2 receptor antagonist and mixtures thereof.

10. A composition of a liquid used for the preparation of a stable slow-release of vitamin B12 orally dissolvable empty capsule shell matrix as in claim 1 wherein the pharmaceutical acceptable excipient is a sweetener(s), a coloring agent(s), a flavoring agent(s), plasticizer(s) and a stabilizer(s).

11. A composition of a liquid used for the preparation of a stable slow-release of vitamin B12 orally dissolvable empty capsule shell matrix as in claim 1 wherein the said capsule is used by (a) placing the said capsule in the mouth cavity between the gum and the cheek; (b) allowing it to dissolve on its own up to 60 minutes without any additional fluid to allow partial or full oral transmucosal absorption of vitamin B12; wherein the capsule comprises a cap and body, each of the cap and body comprising a hard-shell forming material.

12. A stable injectable composition of vitamin B12 comprising: (i) a therapeutic dose of vitamin B12; (ii) optionally a buffer to adjust the pH of the formulation between 4.5 and 7.5: (iii) sodium chloride; (iv) synergistic amounts of a polymer, carrageenan and potassium acetate; (v) hydrochloric acid or sodium hydroxide to adjust the pH between 4.5 and 7.5; (vii) optionally a drug(s) known to impair absorption of vitamin B12; and (viii) water for injection; wherein the solution is prepared under long wavelength light and optionally under nitrogen over-lay; wherein the viscosity of the solution is less than 100 cps.

13. A stable injectable composition of vitamin B12 as in claim 12 wherein vitamin B12 is selected from a group consisting of cyanocobalamin, hydroxocobalamin, adenosylcobalamin, methyl cobalamin, or mixtures thereof.

14. A stable injectable composition of vitamin B12 as in claim 12 wherein the therapeutic dose of vitamin B12 ranges from 0.01 to 10 mg per mL.

15. A stable injectable composition of vitamin B12 as in claim 12 wherein the polymer is selected from hydroxypropyl methylcellulose, hydroxypropyl cellulose, methyl cellulose and mixtures thereof.

16. A stable injectable composition of vitamin B12 as in claim 12 wherein the buffer is a citrate, phosphate, phthalate or an acetate.

17. A stable injectable composition of vitamin B12 as in claim 12 wherein the carrageenan is selected from iota carrageenan, kappa carrageenan, lambda carrageenan and mixtures thereof.

18. A stable nasal spray composition of vitamin B12 comprising: (i) a therapeutic dose of vitamin B12; (ii) synergistic amounts of a polymer, carrageenan and potassium acetate; (iii) optionally a buffer to adjust pH of the formulation between 4.5 and 7.5; (iv) a preservative; (v) a humectant; (vi) purified water; (vii) optionally hydrochloric acid and/or sodium hydroxide to adjust the pH of the formulation between 4.5 and 7.5, and (viii) optionally a drug(s) known to impair absorption of vitamin B12; wherein the solution is prepared under long wavelength light and optionally under nitrogen over-lay wherein the viscosity of the nasal spray is less than 1000 cps.

19. A stable nasal spray composition of vitamin B12 as in claim 18 wherein the therapeutic dose of vitamin B12 ranges from 0.01 to 10 mg per mL.

20. A stable nasal spray composition of vitamin B12 as in claim 18 in which the polymer is selected from hydroxypropyl methylcellulose, hydroxypropyl cellulose, methyl cellulose and mixtures thereof.
